(19) United States
(12) Patent Application Publication
(10) Pub. No.: US 2025/0188437 A1
(43) Pub. Date: Jun. 12, 2025

(54) ENGINEERED ALPHA-GUAIENE SYNTHASES

(71) Applicant: Inscripta, Inc., Pleasanton, CA (US)

(72) Inventors: Karolina Kalbarczyk, Santa Cruz, CA (US); Amanda Reider Apel, Alameda, CA (US)

(21) Appl. No.: 18/838,878

(22) PCT Filed: Feb. 15, 2023
(86) PCT No.: PCT/US2023/062666

§ 371 Date: Aug. 15, 2024

Related U.S. Application Data
(60) Provisional application No. 63/310,976, filed on Feb. 16, 2022.

(51) Int. Cl. C12N 9/88

(52) U.S. Cl. CPC C12N 9/88; C12Y 402/03087

(57) ABSTRACT

The current disclosure relates to compositions and methods relating to engineered polypeptides that can encode guaiene synthases that are selective for alpha-guaiene. The engineered polypeptides can also catalyze the production of increased amounts of alpha-guaiene. The disclosure also relates to host cells including the engineered polypeptides that can be used to produce large quantities of alpha-guaiene and its derivatives. The disclosure also relates to methods of producing alpha-guaiene employing said host cells.

### CROSS-REFERENCE TO RELATED APPLICATION

The present application claims priority to U.S. Provisional patent application Ser. No. 63/310,976, filed on Feb. 16, 2022. The content of the above-referenced application is herein expressly incorporated by reference in its entirety, including any drawings.

### INCORPORATION OF THE SEQUENCE LISTING

The material in the accompanying Sequence Listing is hereby incorporated by reference into this application. The accompanying Sequence Listing file, named 060860-501001WO_Sequence Listing_ST26.xml, was created on Feb. 9, 2023, and is 19,748 bytes.

### BACKGROUND

The guaiene family of plant-derived sesquiterpenes consists of three isomers (alpha, beta, and delta) with qualities that are attractive for consumer applications. Alpha-guaiene is a chemical used in flavorings and fragrances to produce a sweet, woody peppery essence. The production of alpha-guaiene is limited by its presence in rare and sometimes extinct plant species, the difficulty associated with its extraction from such sources and its costly separation from other extraction products.

Alpha-guaiene can be formed in a single step from farnesyl pyrophosphate (FPP) by a terpene synthase (TPS) (Kumeta and Ito, 2010. Characterization of δ-Guaiene Synthases from Cultured Cells of Aquilaria, Responsible for the Formation of the Sesquiterpenes in Agarwood. Plant Physiology, Vol. 154, pp. 1998-2007.) However, most of the isolated and characterized guaiene synthases produce the alpha isomer of guaiene as a minor constituent and in low quantities. This limits their use on an industrial scale.

### SUMMARY

Examples provided here describe methods and compositions of engineered alpha-guaiene synthases and their use to increase titers of alpha-guaiene and its derivatives. In some examples, the compositions described herein may have a benefit of exhibiting an increase in alpha-guaiene titers as well as overall guaiene titers. In some examples, the methods described here may have a benefit of using such alpha-guaiene synthases for large-scale production of alpha-guaiene.

In one aspect of the disclosure, provided herein are engineered polypeptides encoding a guaiene synthase, wherein the engineered polypeptide comprises an amino acid sequence at least about 85% identical to SEQ ID NO:4 and wherein the amino acid sequence comprises a deletion of amino acids corresponding to positions 60-64 of SEQ ID NO:4.

Non-limiting examples of the engineered polypeptides of the disclosure can include engineered polypeptides having an amino acid sequence that is at least about 85%, at least about 90%, at least about 95%, at least about 98% identical to SEQ ID NO:8. In a further aspect, the engineered polypeptide of the disclosure includes an amino acid sequence that is SEQ ID NO:8.

In another aspect, provided herein are nucleic acid molecules encoding the engineered polypeptides of the disclosure. In some aspects, the nucleic acid molecules can include a sequence that is at least about 85% identical, at least about 90%, at least about 95%, at least about 98%, to SEQ ID NO:7. In an aspect, the nucleic acid sequence of the nucleic acid molecule is SEQ ID NO:7.

In one aspect of the disclosure, provided herein are also vectors comprising the nucleic acid molecule of the disclosure.

In a further aspect, provided herein are engineered host cells including the nucleic acid molecule of the disclosure. In an aspect, the host cell may include the vectors of the disclosure.

In some aspects, the host cell is a eukaryotic cell. In some aspects the eukaryotic cell is a fungal cell or a plant cell. In some aspects, the fungal cell is from yeast or from a filamentous fungus. In some aspects, the yeast is from the genus Saccharomyces, Pichia, Yarrowia, or Kluyveromyces. In some aspects, the yeast is Saccharomyces cerevisiae. Or Pichia pastoris, or Yarrowia lipolytica, or Kluyveromyces marxianus. In some aspects, the filamentous fungus is from the genus Rhodosporidium. Aspergillus, Trichoderma, Penicillium. In some aspects, the filamentous fungus is Rhodosporidium toruloides, Aspergillus oryzae, Aspergillus nidulans, Aspergillus niger, Trichoderma reesei, or Penicillium chrysogenum). In further aspects, the host cell is a prokaryotic cell. In some aspects, the prokaryotic cell is from the genus Escherichia, Bacillus, Klebsiella, Lactococcus, Pseudomonas, Mycobacterium, Mannheimia, Corynebacterium, or Vibrio.

In yet another aspect, provided herein are methods of producing, including culturing the host cells of the disclosure wherein the host cell produces total guaiene products comprising alpha-guaiene, wherein the percentage of the alpha-guaiene is at least greater than about 15% of the total guaiene products. In some aspects, the percentage of alpha-guaiene is about 20% to about 99% of the total guaiene products, is about 30% to about 90% of the total guaiene products, is about 40% to about 80% of the total guaiene products, is about 50% to about 70% of the total guaiene products, is about 55% to about 65% of the total guaiene products.

In some aspects of the methods provided herein, the amount of alpha-guanine is at least about 5 g/L or is at least about 4 g/L.

In yet other aspects, provided herein a uses of alpha-guaiene of the disclosure for producing a flavoring or a fragrance constituent. In some aspects, the flavoring or the fragrance constituent is rotundine.

In further aspects, provided herein are methods of producing a flavoring or a fragrance constituent, including culturing the host cells of the disclosure, producing alpha-guaiene; and producing the flavoring or the fragrance constituent from the alpha-guaiene. In some aspects, the host cell produces total guaiene products including alpha-guaiene, wherein the percentage of alpha-guaiene is at least greater than about 15% of the total guaiene products. In some aspects, the flavoring or the fragrance constituent is rotundine.

In other aspects, provided herein are methods for identifying an alpha-guaiene synthase capable of selectively producing alpha-guaiene including selecting an alpha-guaiene synthase comprising a polypeptide sequence; rationally comparing motifs between the polypeptide sequence and at least one other alpha-guaiene synthase from a different species; engineering the polypeptide to add or delete different motifs to obtain an alpha-guaiene synthase that selectively produces more alpha-guaiene than other guaiene products in a host cell and adding or deleting different motifs in order to alter existing enzyme functionality. In some aspects, the engineered polypeptide encodes a guaiene synthase capable of increasing production of alpha-guaiene as compared to a non-engineered polypeptide encoding a guaiene synthase. In some aspects, the engineered polypeptides include a deletion of amino acids corresponding to positions 60-64 of SEQ ID NO:4.

It should be appreciated that all combinations of the foregoing concepts and additional concepts discussed in greater detail below (provided such concepts are not mutually inconsistent) are contemplated as being part of the inventive subject matter disclosed herein and may be employed to achieve the benefits described here. In particular, all combinations of claimed subject matter appearing at the end of this disclosure are contemplated as being part of the inventive subject matter disclosed herein.

The foregoing summary is illustrative only and is not intended to be in any way limiting. In addition to the illustrative implements and features described herein, further aspects, objects and features of the disclosure will become fully apparent from the drawings and the detailed description and the claims.

### BRIEF DESCRIPTION OF THE DRAWINGS

FIG. 1 shows, in one example, the alignment of the first 100 amino acids of the polypeptide sequences of AcC3, SchTPS1 and SchTPS3 guaiene synthases.

FIG. 2, in one example, shows the progression of increasing flux (alpha-guaiene concentration) as the deletions are introduced to SchTPS1 and SchTPS3 synthases in different genetic backgrounds.

FIG. 3 details, in one example, what percent of the final product is alpha-guaiene for the titer data presented in FIG. 2.

FIG. 4 shows, in one example, production of relative high amounts of alpha-guaiene (aggregated data) over time by the highest titer strain STR661 of SEQ ID NO:8 in a scaled-up fermentation process.

FIG. 5 shows, in one example, production of relative high amounts of alpha-guaiene over time by the highest titer strain STR661 of SEQ ID NO:8, separated by replicates. The top graph tracks the percentage of alpha-guaiene in each sample and the bottom graph shows the alpha-guaiene titer for each replicate at each time point.

### DETAILED DESCRIPTION OF THE DISCLOSURE

The current disclosure generally provides example compositions and methods pertaining to engineered polypeptides that can encode guaiene synthases that are selective for alpha-guaiene and/or that produce higher ratios of alpha-guaiene compared to other guaiene isomers. The example engineered polypeptides of the current disclosure can also catalyze the production of increased amounts of alpha-guaiene. The disclosure also provide example host cells including the engineered polypeptides that can be used to produce large quantities of alpha-guaiene and its derivatives. Additional aspects of the disclosure provide example methods of producing alpha-guaiene employing said host cells.

The section headings used herein are for organizational purposes only and are not to be construed as limiting the subject matter described.

### I. General Techniques

The practice of the present disclosure will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003), the disclosures of each of which are incorporated herein by reference in their entirety.

### II. Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

As used herein, when a polypeptide is referred to as having “at least (about) X % sequence identity” to a reference sequence, it is meant that at least (about) X percent of the amino acids in the polypeptide are identical to those of the reference sequence when the sequences are optimally aligned. As used herein, when a nucleic acid is referred to as having “at least X % sequence identity” to a reference sequence, it is meant that at least X percent of the nucleotides are identical to those of the reference sequence when the sequences are optimally aligned.

The term “percent identity,” as used herein in the context of two or more nucleic acid sequences or proteins, refers to two or more sequences or subsequences that are the same or have a specified percentage of nucleotides or amino acids that are the same (e.g., about 50% sequence identity or higher—e.g., about 55%, about 60%, about 65%, about 70%, about 75%, about 80%, about 85%, about 90%, about 91%, about 92%, about 93%, about 94%, about 95%, about 96%, about 97%, about 98%, about 99%, or higher identity over a specified region, when compared and aligned for maximum correspondence over a comparison window or designated region) as measured using, for example The National Center for Biotechnology's (NCBI) BLAST or BLAST 2.0 sequence comparison algorithms with default parameters described below, or by manual alignment and visual inspection. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and publicly available computer programs, such as the GCS program package (Devereux et al, Nucleic Acids Res. 12:387, 1984), BLASTP, BLASTN, FASTA (Atschul et al., J Mol Biol 215:403, 1990 (incorporated herein by reference in its entirety). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. For example, an amino acid sequence that is “substantially identical” to a reference sequence has at least about 50%, about 60%, about 70%, about 75%, about 80%, about 85%, about 90%, about 95%, about 96%, about 97%, about 98%, about 99%, or about 100%, including all values in between, sequence identity to the reference amino acid sequence including all values in between. For polypeptides, the length of comparison sequences will generally be at least 5, at least 6, at least 7, at least 8, at least 9, at least 10, at least 11, at least 12, at least 13, at least 14, at least 15, at least 16, at least 17, at least 18, at least 19, at least 20, at least 25, at least 50, at least 75, at least 90, at least 100, at least 150, at least 200, at least 250, at least 300, or at least 350 contiguous amino acids (e.g., a full-length sequence) including all values in between. For nucleic acids, the length of comparison sequences will generally be at least 5, at least 10, at least 11, at least 12, at least 13, at least 14, at least 15, at least 16, at least 17, at least 18, at least 19, at least 20, at least 21, at least 22, at least 23, at least 24, or 25 contiguous nucleotides including all values in between (e.g., the full-length nucleotide sequence).

The term “engineered” or “recombinant” polypeptide as used herein, refers to a polypeptide that has been altered through human intervention. As non-limiting examples, an engineered polypeptide can be one which: 1) has been synthesized or modified in vitro, for example, using chemical or enzymatic techniques; 2) includes conjoined polypeptide sequences that are not conjoined in nature; 3) has been engineered using molecular cloning techniques such that it lacks one or more amino acids with respect to the naturally occurring polypeptide sequence; and/or 4) has been manipulated using molecular cloning techniques such that it has one or more sequence changes or rearrangements with respect to the naturally occurring polypeptide.

A “vector” or “expression vector” is a replicon, such as plasmid, phage, virus, or cosmid, to which another nucleic acid segment, i.e., an “insert”, may be attached so as to bring about the replication and/or expression of the attached segment in a cell.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some aspects, the term “about” indicates the designated value±up to 10%, up to ±5%, or up to ±1%, and in some instances±0% for exact value as described above.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable sub-combination. All combinations of the aspects pertaining to the disclosure are specifically embraced by the present disclosure and may be used to achieve the benefits described herein and are disclosed herein just as if each and every combination was individually and explicitly disclosed. In addition, all sub-combinations of the various aspects and elements thereof are also specifically embraced by the present disclosure and are disclosed herein just as if each and every such sub-combination was individually and explicitly disclosed herein.

### III. Compositions

The compositions disclosed herein include engineered polypeptides that encode a guaiene synthase capable of increasing production of alpha-guaiene as compared to a non-engineered polypeptide encoding a guaiene synthase. In some aspects, the engineered polypeptides comprise a deletion of amino acids at positions 60-64 of SEQ ID NO:4.

The compositions disclosed herein also include engineered polypeptides that encode a guaiene synthase, wherein the engineered polypeptides have amino acid sequences with substantial identity or a certain sequence identity to SEQ ID NO:4 and wherein the amino acid sequence comprises a deletion of amino acids at positions 60-64 of SEQ ID NO:4. SEQ ID NO:4 is a SchTPS3 guaiene synthase from Stellera chamaejasme L.

In some aspects of the disclosure, the engineered polypeptide can have at least about 85%, about 86%, about 87%, about 88%, about 89%, about 90%, about 91%, about 92%, about 93%, about 94%, about 95%, about 96%, about 97%, about 98%, about 99% or about 100% sequence identity, including all values in between, to SEQ ID NO: 4 with a deletion of amino acids at positions 60-64 of SEQ ID NO:4.

The compositions disclosed herein also include nucleic acid molecules encoding the engineered polypeptides of the disclosure. In some aspects, the nucleic acid molecules encode a guaiene synthase capable of increased production of alpha-guaiene synthase as compared to a non-engineered polypeptide encoding a guaiene synthase. In some aspects, the nucleic acid molecule includes a sequence that may be substantially identical to SEQ ID NO: 7. In some aspects, the nucleic acid sequence may be at least about 85% identical to SEQ ID NO: 7.

In some aspects of the disclosure, the nucleic acid molecules can have at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% sequence identity including all values in between to SEQ ID NO:7.

An optimal alignment of sequences can be determined in various ways that are within the skill in the art, for instance, the Smith Waterman alignment algorithm (Smith T F et al., J. Mol. Biol. 1981; 147:195-7) and BLAST (Basic Local Alignment Search Tool; Altschul S F et al., J. Mol. Biol. 1990; 215:403-10). These and other alignment algorithms are accessible using publicly available computer software such as “Best Fit” (Smith T F et al., Adv. Appl. Math. 1981; 2 (4): 482-9) as disclosed for GeneMatcher Plus™ (Schwarz and Dayhof, “Atlas of Protein Sequence and Structure,” ed. Dayhoff, M. O., pp. 353-358, 1979), BLAST, BLAST-2, BLAST-P, BLAST-N, BLAST-X, WU-BLAST-2, ALIGN, ALIGN-2, CLUSTAL, T-COFFEE, MUSCLE, MAFFT, or Megalign (DNASTAR). In addition, those skilled in the art can determine appropriate parameters for measuring alignment, including any algorithms needed to achieve optimal alignment over the length of the sequences being compared. In general, for polypeptides, the length of comparison sequences can be at least five amino acids, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 150, at least 175, at least 200, at least 250, at least 300, at least 400, at least 500 or more amino acids, up to the entire length of the polypeptide and including all values in between. For nucleic acids, the length of comparison sequences can generally be at least 10, at least 20, at least 30, at least 40, at least 50, at least 60, at least 70, at least 80, at least 90, at least 100, at least 125, at least 150, at least 175, at least 200, at least 250, at least 300, at least 400, at least 500, at least 600, at least 700, at least 800, at least 900, at least 1000, at least 1100, at least 1200, at least 1300, at least 1400, at least 1500, at least 1600, or more nucleotides, up to the entire length of the nucleic acid molecule including all values in between.

### Guaiene Synthases

Provided herein are engineered polypeptides encoding guaiene synthases. Guaiene synthases are sesquiterpene synthases known as the rate-limiting enzymes in many biosynthetic pathways. High levels of production often involves a high-copy plasmid expression or many genomic copies of the synthase, which can lead to genetic instability of the cell lineage. Therefore, there it can be beneficial to identify guaiene synthases with improved kinetics, so that fewer copies of synthase yield similar titers thereby reducing instability and preventing toxic buildup of metabolic intermediates. Examples of this disclosure provides example engineered alpha-guaiene synthase enzymes.

Alpha-guaiene synthases can catalyze the formation of alpha-guaiene. In some aspects, the alpha-guaiene synthase is a farnesyl-diphosphate diphosphate-lyase enzyme that can use (2E,6E)-farnesyl diphosphate as a substrate. In some aspects, the alpha-guaiene can catalyze the following chemical reaction:

(2E,6E)-farnesyl diphosphate→alpha-guaiene+diphosphate

Non-limiting examples of alpha-guaiene synthases include UniProtKB Identifier: DOVMR7, UniProtKB Identifier: DOVMR8, UniProtKB Identifier: DOVMR6, UniProtKB Identifier: Q49SP3.

In certain aspects, an alpha-guaiene synthase may be capable of producing guaiene products that includes at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% including any values in between, alpha-guaiene.

In some examples, an alpha-guaiene synthase is capable of producing guaiene products that includes between 1% to 10%, between 5% to 20%, between 15% to 20%, between 16% and 20%, between 17% and 20%, between 18% and 20%, between 19% and 20%, between 20% and 25%, between 20% and 24%, between 20% and 23%, between 20% and 22%, between 20% and 21%, between 20% and 30%, between 30% and 40%, between 40% and 50%, between 50% and 60%, between 60% and 70%, between 70% and 80%, between 80% and 90%, or between 90% and 100%, including any values in between, alpha-guaiene.

As disclosed herein, an alpha-guaiene synthase may include a polypeptide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more than 99%, including all values in between, identical to SEQ ID NO:6 or to SEQ ID NO:8. In certain aspects, an alpha-guaiene synthase comprises SEQ ID NO:6 or SEQ ID NO:8. In certain aspects, an alpha-guaiene synthase consists of SEQ ID NO:6 or consists of SEQ ID NO:8.

In one aspect, the alpha-guaiene synthase may have least 2-fold increase in activity, such as 3-fold activity, 4-fold activity, 5-fold activity, 6-fold activity, 7-fold activity, 8-fold activity, 9-fold activity, 10-fold activity, 11-fold activity, 12-fold activity, 13-fold activity, 14-fold activity, 15-fold activity, 16-fold activity, 17-fold activity, 18-fold activity, 19-fold activity, 20-fold activity, 21-fold activity, 22-fold activity, 23-fold activity, 24-fold activity, 25-fold activity, or any other value in between, when compared with other alpha-guaiene synthases.

As disclosed herein, an alpha-guaiene synthase may be encoded by a nucleotide sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more than 99%, including all values in between, identical to SEQ ID NO:5 or to SEQ ID NO:7. In certain aspects, an alpha-guaiene synthase may be encoded by a sequence comprising SEQ ID NO:5 or SEQ ID NO:7. In certain aspects, an alpha-guaiene synthase may be encoded by SEQ ID NO:5 or by SEQ ID NO:7.

### Vectors

In some aspects, the current disclosure pertains to vectors including the nucleic acid molecules described herein or to expression vectors including the engineered polypeptides of the disclosure.

Vectors can either replicate in the cytoplasm of the host microorganism or integrate into the chromosomal DNA of the host microorganism. In either case, the vector can be a stable vector (e.g., the vector remains present over many cell divisions, even if only with selective pressure) or a transient vector (e.g., the vector is gradually lost by host microorganisms with increasing numbers of cell divisions).

A vector encoding any of the guaiene synthases described herein may be introduced into a suitable host cell using any suitable method. Non-limiting examples of yeast transformation protocols are described for example, in Gietz et al., Yeast transformation by the LiAc/SS Carrier DNA/PEG method. Methods Mol. Biol. 2006; 313:107-20.

### Host Cells

In another aspect, provided herein are host cells including the engineered polypeptides, the nucleic acid molecules and/or the vectors of the disclosure.

Any suitable host cell may be used to produce any of the guaiene synthase engineered polypeptides disclosed herein, including prokaryotic cells or eukaryotic cells. Suitable prokaryotic cells include bacteria cells, for example, but are not limited to bacterial cells from the genus Escherichia Bacillus, Klebsiella, Lactococcus, Pseudomonas, Mycobacterium, Mannheimia, Corynebacterium, and Vibrio.

Suitable eukaryotic host cells include fungal cells (e.g., yeast cells or filamentous fungal cells and others). Non-limiting examples of genera of yeast for expression include Saccharomyces (e.g., Saccharomyces cerevisiae), Pichia (e.g., Pichia pastoris), Kluyveromyces (e.g. Kluyveromyces marxianus), Hansenula and Yarrowia (e.g. Yarrowia lipolytica). In some aspects, the yeast strain is an industrial polyploid yeast strain. Non-limiting examples of filamentous fungal cells include cells obtained from Aspergillus (e.g., Aspergillus oryzae, Aspergillus nidulans, Aspergillus niger) or Trichoderma (e.g. Trichoderma reesei), or Penicillium (e.g., Penicillium chrysogenum), or Rhodosporidium (e.g., Rhodosporidium toruloides). Any of the aforementioned host cells described may be suitable to achieve the benefits described herein.

Host cells may be cultured under any conditions suitable for the type of cell involved. For example, any suitable media, temperatures, and incubation conditions may be used. For host cells carrying an inducible vector, cells may be cultured with an appropriate inducible agent to promote expression. Any suitable techniques for transforming the above-mentioned host cells and species may be used.

Host cells may be cultured in flasks, plates, and bioreactors. In an aspect, standard yeast cultivation conditions are used, such as for example with a temperature at about 30° C. and varying levels of agitation. For shake flasks, non-limiting examples of agitation rates may be between about 300 rpm and about 400 rpm. In 96 well plate cultivation, the plates may be sealed with a porous seal (to allow oxygen for the cells) and the growth may occur at 30° C. and 1000 rpm, with 80% humidity. Agitation rates for different shakers may need to be identified empirically.

Cell cultures including at least one host cell and a culture medium as disclosed herein are also within the scope of this application. Generally, the culture medium can be any suitable culture medium for culturing the cells described herein. Examples of methods and systems suitable for generating and maintaining cell cultures that can be used include, but is not limited to, “Culture of Animal Cells—A Manual of Basic Technique” by Freshney, Wiley-Liss, N.Y. (1994), Third Edition and its updated editions). The skilled artisan will recognize that such conditions can be modified to accommodate different microorganisms.

Any of the cells disclosed herein can be cultured in media of any type (rich or minimal) and any composition prior to, during, and/or after contact and/or integration of a nucleic acid. The conditions of the culture or culturing process can be optimized through routine experimentation as understood by one of ordinary skill in the art. In some aspects, the selected media is supplemented with various components. In some aspects, the concentration and amount of a supplemental component is optimized. In some aspects, other elements of the media and growth conditions (e.g., pH, temperature, etc.) are optimized through routine experimentation. In some aspects, the frequency that the media is supplemented with one or more supplemental components, and the amount of time that the cell is cultured is optimized.

Culturing of the cells described herein can be performed in culture vessels known and used in the art. In some aspects, an aerated reaction vessel (e.g., a stirred tank reactor) is used to culture the cells. In some aspects, a bioreactor or fermentor is used to culture the host cells of the disclosure. Thus, in some aspects, the host cells are used in fermentation. As used herein, the terms “bioreactor” and “fermentor” are interchangeably used and refer to an enclosure, or partial enclosure, in which a biological, biochemical and/or chemical reaction takes place, involving a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: bioreactors agitated by rotating mixing devices, chemostats, bioreactors agitated by shaking devices, airlift fermentors, stirred tank fermentors, fluidized bed bioreactors, bioreactors employing wave induced agitation, centrifugal bioreactors, roller bottles, hollow fiber bioreactors, packed-bed reactors, fixed-bed reactors, roller apparatuses, vertically-stacked plates, spinner flasks, stirring or rocking flasks, shaken multi-well plates, MD bottles, T-flasks, Roux bottles, multiple-surface tissue culture propagators, modified fermentors, coated beads (e.g., beads coated with serum proteins, nitrocellulose, or carboxymethyl cellulose to prevent cell attachment) and others as known to a person of skill in the art.

In some aspects, the bioreactor includes a cell culture system where the cell (e.g., yeast cell) is in contact with moving liquids and/or gas bubbles. In some aspects, the cell or cell culture is grown in suspension. In other aspects, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some aspects, carriers are fabricated from materials such as dextran, gelatin, glass, or cellulose.

### IV Methods of the Disclosure

The discussion of the general methods given herein is intended for illustrative purposes only. Other alternative methods and alternatives will be apparent to those of skill in the art upon review of this disclosure, and are to be included within the spirit and purview of this application.

### Methods for Producing Alpha-Guaiene

Provided herein are example methods of producing alpha-guaiene including culturing the host cell of the disclosure, wherein the host cell produces guaiene products comprising alpha-guaiene, wherein the alpha-guaiene is in a greater amount than the other guaiene products. Examples of other guaiene products include but are not limited to other guaiene isomers such as beta-guaiene and/or delta-guaiene. In some examples, the host cell produces alpha-guaiene at a higher percentage of total guaiene (i.e., combined total of all of the guaiene isomers, such as alpha, beta, and delta isomers) than what the host cell produces of the beta-guaiene as a percentage of the total guaiene. In some examples, the host cell produces alpha-guaiene at a higher percentage of total guaiene than what the host cell produces of the delta-guaiene as a percentage of the total guaiene.

Also provided herein are example methods of producing alpha-guaiene including culturing the host cell of the disclosure, wherein the host cell produces more alpha-guaiene than any other guaiene product produced by the host cell.

In some aspects, provided herein are methods of producing alpha-guaiene including culturing any of the host cells of the disclosure, wherein the host cell produces guaiene products comprising alpha-guaiene, wherein the percentage of alpha-guaiene is at least greater than 15% of the total guaiene products. In some aspects of the methods of the disclosure, the percentage of alpha-guaiene may be at least greater than about 15% of the total guaiene products, at least greater than about 16%, at least greater than about 17%, at least greater than about 18%, at least greater than about 19%, at least greater than about 20%, at least greater than about 21%, at least greater than about 25%, at least greater than about 30%, at least greater than about 35%, at least greater than about 40%, at least greater than about 45%, at least greater than about 50%, at least greater than about 55%, at least greater than about 60%, at least greater than about 65%, at least greater than about 70%, at least greater than about 75%, at least greater than about 80%, at least greater than about 85%, at least greater than about 90%, at least greater than about 95%, at least greater than about 99%, or any values in between, of the total guaiene products.

In some aspects of the methods of the disclosure, the percentage of alpha-guaiene may be greater than about 15%, greater than about 16%, greater than about 17%, greater than about 18%, greater than about 19% greater than about 20%, greater than about 21%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95%, greater than about 99%, or any values in between of the total guaiene products.

In some aspects of the methods of the disclosure, the percentage of alpha-guaiene may be at least about 15% of the total guaiene products, at least about 16%, at least about 17%, at least about 18%, at least about 19%, at least about 20%, at least about 21%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or any values in between, of the total guaiene products.

In some aspects of the methods of the disclosure, the percentage of alpha-guaiene may be about 15% of the total guaiene products. In some aspects, the percentage of alpha-guaiene may be about 16%, about 17%, about 18%, about 19%, about 20%, about 21%, about 22%, about 23%, about 24%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or any values in between of the total guaiene products.

In some aspects of the methods of the disclosure the percentage of alpha-guaiene may be about 15% to about 99% of the total guaiene products. In some aspects the percentage of alpha-guaiene to the total guaiene products may be about 20% to about 97%, about 25% to about 95%, about 30% to about 90%, about 35% to about 85%, about 40% to about 80%, about 45% to about 75%, 50% to about 70%, about 55% to about 65%, or any ranges in between, of the total guaiene products. Other ranges are also possible.

In some aspects of the methods of the disclosure, the amount of alpha-guaiene that is produced by the methods is at least about 2.0 g/L, at least about 2.5 g/L, at least about 3.0 g/L, at least about 3.5 g/L, at least about 4.0 g/L, at least about 4.5 g/L, at least about 5.0 g/L, at least about 5.5 g/L, at least about 6.0 g/L, at least about 6.5 g/L, at least about 7.0 g/L or higher.

In some aspects of the methods of the disclosure, the amount of alpha-guaiene that is produced by the method is at greater than about 1.0 g/L, greater than about 1.5 g/L, greater than about 2.0 g/L, greater than about 2.5 g/L, greater than about 3.0 g/L, greater than about 3.5 g/L, greater than about 4.0 g/L, greater than about 4.5 g/L, greater than about 5.0 g/L, greater than about 5.5 g/L, greater than about 6.0 g/L, greater than about 6.5 g/L, greater than about 7.0 g/L or higher.

In some aspects of the methods of the disclosure, the amount of alpha-guaiene that is produced by the method is about 1.0 to about 7.0 g/L, about 1.5 g/L to about 6.5 g/L, about 2.0 to about 6.0 g/L, about 2.5 g/L to about 5.5 g/L, about 3.0 g/L to about 5.0 g/L, about 3.5 g/L to about 4.5 g/L. Other ranges, including any values in between, are also possible.

The amount of alpha-guaiene may be assayed by any suitable method or assay, including but not limited to Gas Chromatography-Mass Spectrometry (GCMS).

Methods for Producing a Flavoring or a Fragrance Constituent from Alpha-Guaiene

Also provided herein are methods of producing a flavoring or a fragrance constituent including culturing a host cell of the disclosure, producing alpha-guaiene and producing the flavoring or the fragrance constituent.

In some aspects of the methods of producing a flavoring or a fragrance constituent, the host cell may produce guaiene products comprising alpha-guaiene, wherein the percentage of alpha-guaiene is at least greater than about 15% of the total guaiene products. In some aspects of the methods of the disclosure, the percentage of alpha-guaiene may be at least greater than about 15% of the total guaiene products, at least greater than about 16%, at least greater than about 17%, at least greater than about 18%, at least greater than about 19%, at least greater than about 20%, at least greater than about 21%, at least greater than about 25%, at least greater than about 30%, at least greater than about 35%, at least greater than about 40%, at least greater than about 45%, at least greater than about 50%, at least greater than about 55%, at least greater than about 60%, at least greater than about 65%, at least greater than about 70%, at least greater than about 75%, at least greater than about 80%, at least greater than about 85%, at least greater than about 90%, at least greater than about 95%, at least greater than about 99%, or any values in between, of the total guaiene products.

In some aspects, the flavoring or the fragrance constituent may be rotundine. Rotundine is a sesquiterpene that is present in the tubers of Java grass (Cyperus rotundus). Rotundone is also present in the essential oils of white and black pepper, marjoram, oregano, rosemary, basil, thyme, and geranium, as well as in some Syrah wines. It is also present in agarwood, cypriol oil and patchouli oil. It can impart flavorings or fragrances with a sweet, woody peppery essence or aroma.

In some aspects, the flavoring constituents of the disclosure may enhance the flavors of foods and/or drinks. Non-limiting examples of foods include processed foods such as stews, soups, noodles, sauces, dairy-based foods (such as ice-cream), chewing gum, chocolate, candy, seasonings and various others. Non-limiting examples of the drinks may include chocolate-flavored drinks, tea-based drinks, beer-based drinks, dairy-based drinks, coffee-based drinks and various others.

In some aspects, the fragrance constituents of the disclosure may be included in essential oils, soaps, cosmetics, creams, fragrances, candles, and various other products.

### Methods for Identifying Guaiene Synthases

Also provided herein are example methods of identifying guaiene synthases capable of selectively producing alpha-guaiene. Selective production can mean that alpha-guaiene is produced by the engineered host cell(s) in greater amounts as compared to other isomers, for example beta-guaiene or delta-guaiene.

In some aspects, provided herein are methods for identifying an alpha-guaiene synthase capable of selectively producing alpha-guaiene including selecting an alpha-guaiene synthase having a polypeptide sequence, rationally comparing motifs between the polypeptide sequence and at least one other alpha-guaiene synthase from a different species, engineering the polypeptide to add or delete different motifs to obtain an alpha-guaiene synthase that selectively produces more alpha-guaiene than other guaiene products in a host cell, and adding or deleting or modifying different motifs in order to alter existing enzyme functionality.

The amount of alpha-guaiene may be assayed by any suitable method or assay. For example, alpha-guaiene may be quantitated by GCMS.

An example of a workflow to identify guaiene synthases capable of selectively producing alpha-guaiene and/or producing alpha-guaiene in relatively high amounts than other guaiene products produced by a host cell is described herein for illustrative purposes and is not to be interpreted as having a limited scope. An alpha-guaiene synthase enzyme of interest may be selected and its amino acid sequence sourced from a polypeptide sequence database such as Uniprot. The enzyme may be codon optimized for expression in a target host such as, for example, Saccharomyces cerevisiae. The polypeptide sequence of interest may be aligned with a reference sequences such as SEQ ID NO:8 and/or SEQ ID NO:4 to rationally compare motifs and structural variations. Any suitable method for aligning polypeptide sequences may be used. If the polypeptide sequence of interest includes additional amino acids present at amino acids 60-64 which are not present in SEQ ID NO:8 and/or SEQ ID NO:4, then deleting said amino acids. The polypeptide of interest may be from a different species than the reference polypeptide, SEQ ID NO:8, and/or SEQ ID NO:4. One or more copies of the truncated enzyme may be built into suitable strain backgrounds of host cells, such as those of Table 1, and assessed for the production of alpha-guaiene in a host cell culture by any suitable assay. The percentage of produced alpha-guaiene may be compared to the percentage of the total guaiene products produced by the host cell.

In certain aspects, an alpha-guaiene produced may be at least 15%, at least 16%, at least 17%, at least 18%, at least 19%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% including any values in between, of the total guaiene products.

In some examples, the alpha-guaiene produced may be between 1% to 10%, between 5% to 20%, between 15% to 20%, between 16% and 20%, between 17% and 20%, between 18% and 20%, between 19% and 20%, between 20% and 25%, between 20% and 24%, between 20% and 23%, between 20% and 22%, between 20% and 21%, between 20% and 30%, between 30% and 40%, between 40% and 50%, between 50% and 60%, between 60% and 70%, between 70% and 80%, between 80% and 90%, or between 90% and 100%, including any values in between, of the total guaiene products produced by the host cell.

In one aspect, the modifications described herein for engineering the alpha-guaiene polypeptide sequence may result in at least 2-fold increase in activity, such as 3-fold activity, 4-fold activity, 5-fold activity, 6-fold activity, 7-fold activity, 8-fold activity, 9-fold activity, 10-fold activity, 11-fold activity, 12-fold activity, 13-fold activity, 14-fold activity, 15-fold activity, 16-fold activity, 17-fold activity, 18-fold activity, 19-fold activity, 20-fold activity, 21-fold activity, 22-fold activity, 23-fold activity, 24-fold activity, 25-fold activity, or any other value in between, of the alpha-guaiene enzyme.

### Uses of Alpha Guaiene

Also provided herein are uses of the methods of the disclosure or uses of the alpha-guaiene produced by the methods of the disclosure to produce flavoring or fragrance constituents. In an aspect, the flavoring or fragrance constituents may be rotundone. The methods of the disclosure circumvent the need to extract rotundone from plant material, and the alpha-guaiene required may be furnished in a stable quality and in the substantial absence of impurities.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application. It is to be understood that various modifications and combinations are possible and are contemplated within the true spirit and scope of the appended claims.

TABLE 1
S. cerevisiae Strains Described in
the Drawings of the Current Disclosure

| Label | Genotype |
|---|---|
| CEN.PK | Non producer |
| STR063 | Low flux terpene strain |
| STR247 | Medium flux terpene strain |
| STR289 | Low flux strain background + SchTPS3 |
| STR295 | Low flux strain background + SchTPS1 |
| STR363 | Low flux strain background + SchTPS1 with deletion |
| STR515 | Low flux strain background + SchTPS3 with deletion |
| STR630 | Medium flux strain background + SchTPS3 |
| STR631 | Medium flux strain background + SchTPS1 with deletion |
| STR639 | Medium flux strain background + SchTPS3 with deletion |
| STR659 | High flux strain background + SchTPS3 with deletion |
| STR661 | High flux strain background + 2 copies of SchTPS3 |
| | with deletion |

Additional aspects are disclosed in further detail in the following examples, which are provided by way of illustration and are not in any way intended to limit the scope of this disclosure or the claims.

### NON-LIMITING WORKING EXAMPLES

The following working examples outline the different steps of an example engineering of an alpha-guaiene synthase that when expressed in a host cell causes the selective production of higher amounts of alpha-guaiene as compared to other guaiene products produced by the host cell.

### Example 1—Alignment of Sequences and Codon Optimization

This examples demonstrates how homologous polypeptides of alpha-guaiene synthases of interest may be selected and analyzed for structural variations. SchTPS1 and SchTPS3 enzymes were selected based upon initial reports of guaiene production (An et al. 2020). Amino acid sequences for both enzymes were sourced from Uniprot and the enzymes were codon optimized for expression in Saccharomyces cerevisiae. Codon optimized SchTPS1 and SchTPS3 were individually integrated into the Gal80 locus driven by pGAL1 and tGAT2.

After initial validation of activity in the SchTPS1 & SchTPS3 enzymes, the two protein structures were aligned to compare possible structural variations. This practice of aligning homologous proteins has been widely documented (Rost, 1999; Wang, 2018; Wiltgen, 2019).

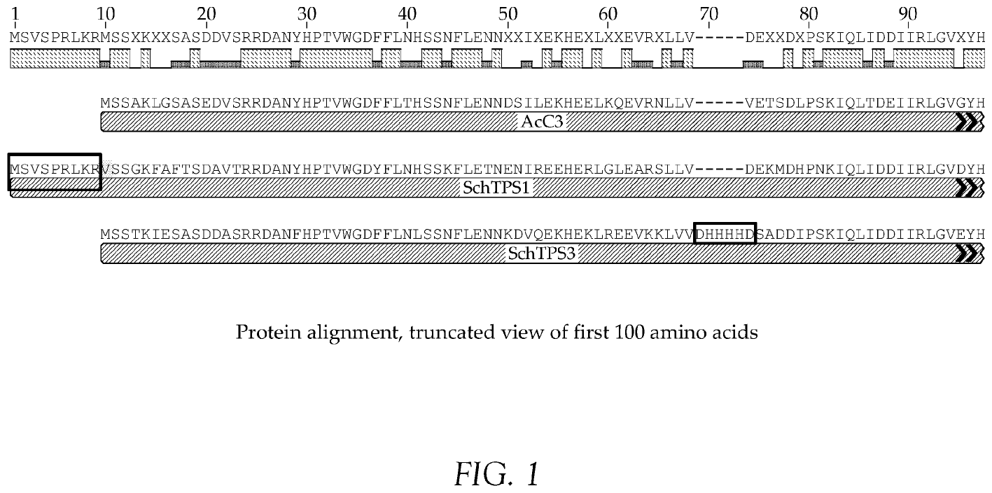

Two differences between the proteins were selected for further investigation (visible in the truncated alignment presented in FIG. 1): (1) SchTPS1 had 9 amino acids present at the N-terminus of the protein which were not present in SchTPS3 and (2) SchTPS3 had 5 amino acids present at amino acids 60-64 which were not present in SchTPS1. The sequences were engineered as follows in Example 2.

### Example 2—Engineering SCHTPS1 and SCHTPS3 from Stellera chamaejasme L

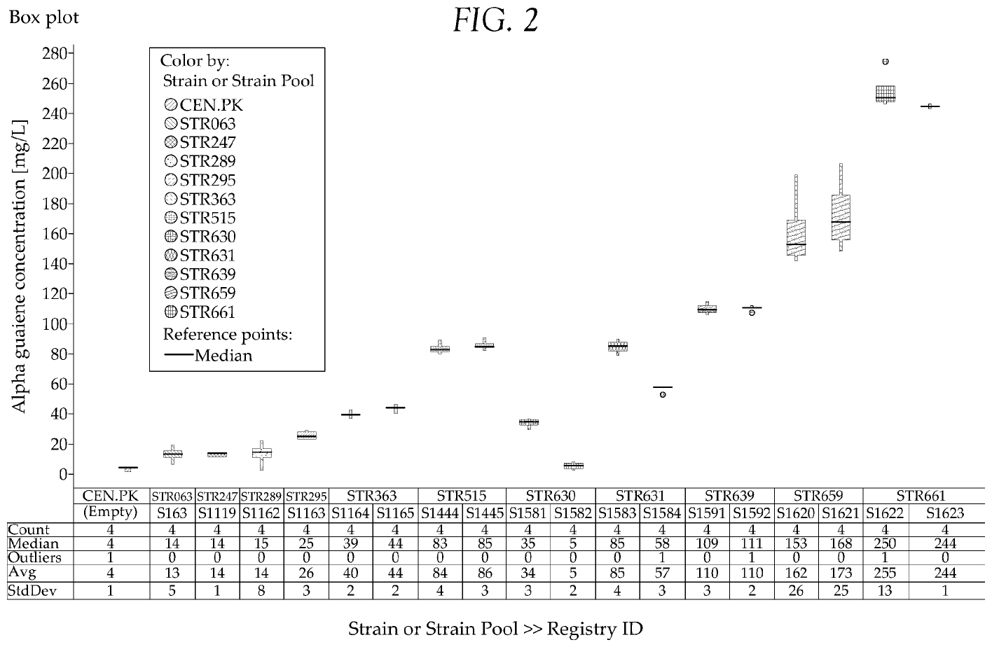

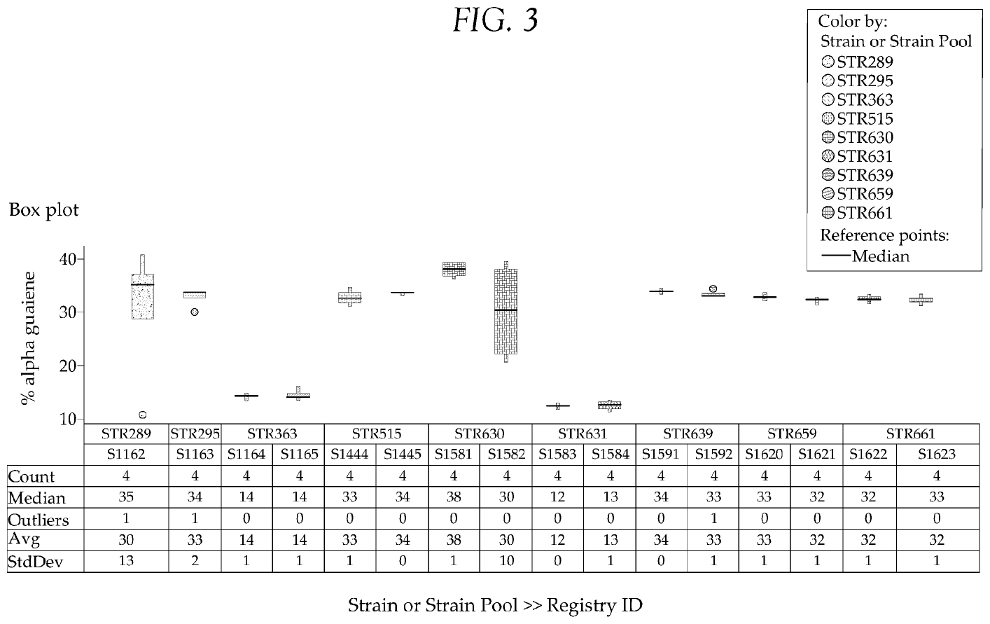

This example demonstrates the engineering of a guaiene synthase of interest. The 9 amino acids at the front of SchTPS1 were replaced with a methionine, and the 5 amino acids in SchTPS3 were deleted. These truncated enzymes were built into the initial proof-of-concept strains, and further built into more advanced strain backgrounds developed for higher terpene flux. Two copies of the SchTPS3 enzyme with the 5 amino acid deletion were built into different background strains described in Table 1 to achieve high flux to the desired alpha-guaiene compound. Compared to the initial assessment introducing the SchTPS3 to the low flux strain, modifying the enzyme and introducing this to a higher flux background resulted in more than 16-fold increase in activity in the plate-based activity assessment as shown in FIGS. 2 and 3.

### Example 3—Engineered Guaiene Synthases are Alpha-Selective and High Producers

FIG. 2 shows the progression of increasing flux as the deletions are introduced to the SchTPS1 & SchTPS3 synthases, and these synthases are introduced to higher flux lineages. All data presented in FIGS. 2 and 3 were obtained through cultivation of cells in a 96 well plate. In the low flux background, SchTPS3 with a five amino acid deletion resulted in a ˜5.5-fold titer increase (comparing STR289 to STR515) and SchTPS1 with a nine amino acid deletion resulted in a ˜1.6-fold titer increase (comparing STR295 to STR363.)

In a medium flux background, SchTPS3 with a five amino acid deletion again resulted in a ˜5.5-fold titer increase (comparing STR630 to STR639). The SchTPS3 with a five amino acid deletion increased titer when introduced to the high flux background (comparing STR639 to STR659), with highest titer identified with two copies of the highest performing synthase in the high flux background strain.

FIG. 3 details what percent of the final product is the desired alpha-guaiene compound. The strains with SchTPS1 with the nine amino acid deletion (STR363 and STR631) demonstrate that while titer was increased in these samples (shown in FIG. 2), the specific percentage of alpha-guaiene was decreased. The SchTPS3 with five amino acids deleted resulted in increased titers (shown in FIG. 2) but retained a higher percentage of the desired alpha-guaiene product (comparing STR289 and STR515, STR630 and STR639). This data illustrates the value of the SchTPS3 with the five amino acid deletion, which increased production of alpha-guaiene while retaining selectivity for the desired product.

### Example 4—Large Scale Production of Alpha-Guaiene

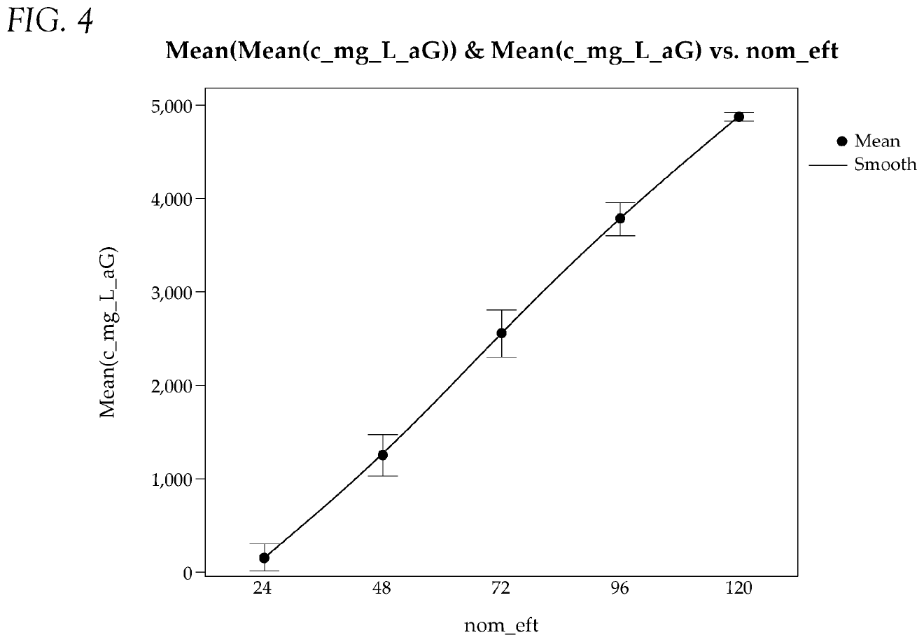

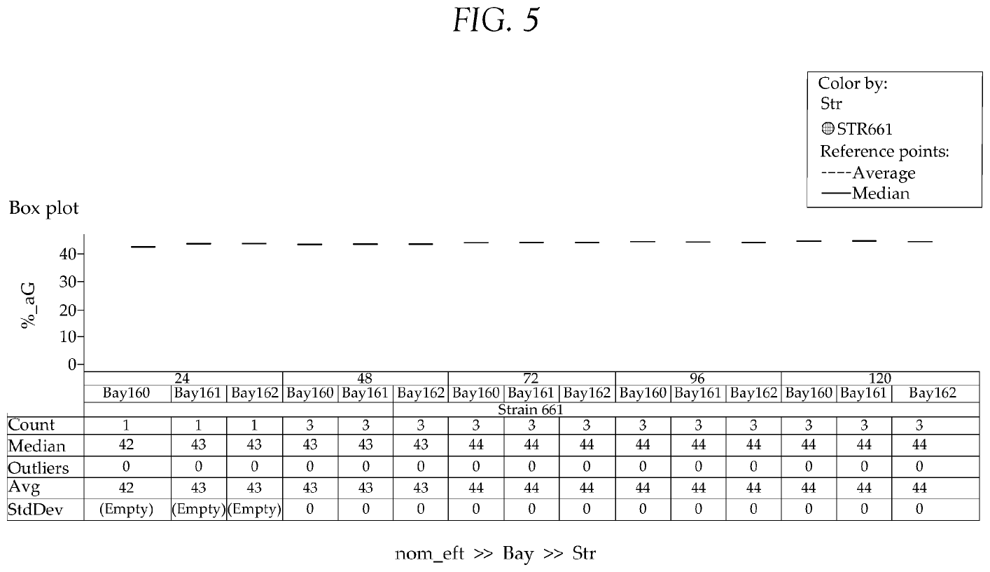

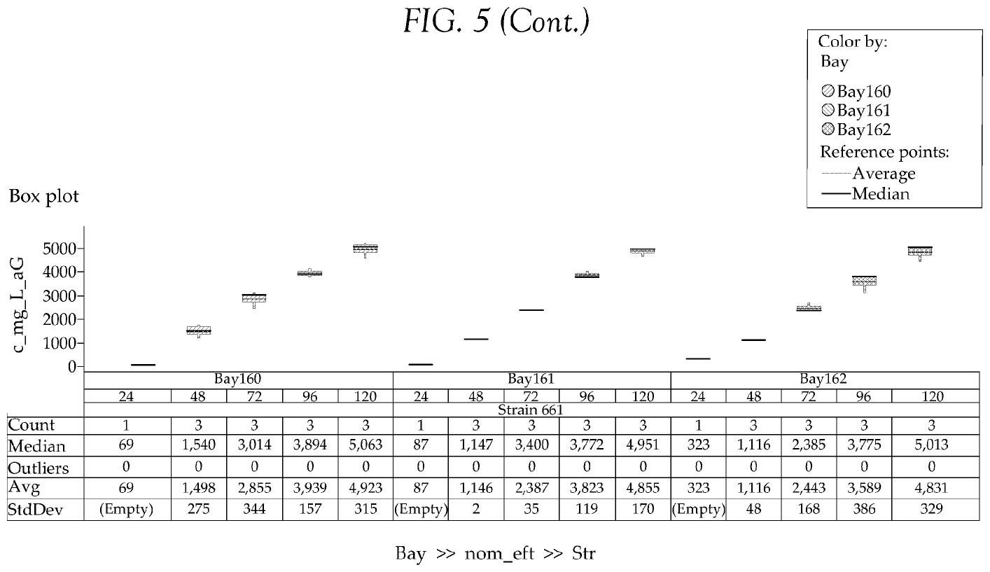

To assess the scalability of this discovery, the highest titer strain in plates carrying the engineered SchTPS3 with five amino acids deleted (STR661) was run in three, 250 mL bioreactors for 120 hours. Production of alpha-guaiene reached 5 g/L by 120 hours and represented over 40% of the total guaiene output of 12 g/L (FIG. 4 aggregated; FIG. 5 individual reactors titer bottom with alpha percentage top.)

### Example 5—Quantitating Alpha-Guaiene

Alpha-guaiene was quantitated via Gas Chromatography-Mass Spectrometry (GCMS). Experimental samples, generated via fermentation or micro-fermentation, were diluted with isopropyl alcohol. Samples were analyzed on an Agilent 8890 GC with a 7010 Mass Spectrometer using a PAL RSI 120 autosampler. 1 μL of each sample was injected onto a DB-SELECT 624 UI 60 m 0.250 mm ID 1.40 micron film column via a split/splitless inlet at a 3:1 split. The column was run with a constant flow at 1.2 mL/min. The temperature was held at 80° C. for 1 minute, then ramped to 250° C. at 10° C./min followed by a hold at 250° C. The MS source was run in an electron ionization mode at 230° C., and 200 ms scans were run from 40-300 m/z. An authentic standard of alpha-guaiene at 5 mg/mL in chloroform was acquired from Cayman chemical and used to identify the correct target peak, and quantitate via an external standard curve method. Alpha-guaiene peak identity was further confirmed for both samples and standard via search against The National Institute of Standards and Technology (NIST) Mass Spectrometry (MS) database.

### Additional Notes

It is appreciated that certain features of the disclosure, which are, for clarity, described in the context of separate aspects, can also be provided in combination in a single implement. Conversely, various features of the disclosure, which are, for brevity, described in the context of a single implement, can also be provided separately or in any suitable sub-combination. All combinations of the aspects pertaining to the disclosure are specifically embraced by the current disclosure and are disclosed herein just as if each and every combination was individually and explicitly disclosed. In addition, all sub-combinations of the various aspects and elements thereof are also specifically embraced by the current disclosure and are disclosed herein just as if each and every such sub combination was individually and explicitly disclosed herein.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

### INFORMAL SEQUENCE LISTING

| SEQ | | |
|---|---|---|
| ID | | |
| NO | Sequence | Description |
| 1 | ATGAGTGTATCTCCGAGGTTAAAGAGAGTA | SchTPS1 DNA |
| | TCCTCTGGAAAGTTCGCCTTTACATCAGAT | Sequence |
| | GCAGTTACTAGAAGAGATGCTAATTACCAT | |
| | CCAACCGTCTGGGGTGATTATTTTCTAAAC | |
| | CATTCTAGTAAATTCTTAGAAACTAATGAA | |
| | AACATTAGAGAAGAACACGAAAGGTTGGGT | |
| | CTTGAAGCAAGATCCCTGTTGGTAGATGAG | |
| | AAAATGGATCACCCAAACAAAATTCAATTG | |
| | ATTGATGACATTATTAGGCTTGGGGTCGAT | |
| | TACCATTTTGAATCGGAAATCAACGCTGAA | |
| | CTAAAGAAACTGCATGATGACCACGACCAC | |
| | TTAGACTTCGATTTAAGAACCACTTCCACT | |
| | TTGTTCAGATTACTAAGAGCACATGGTTTT | |
| | CAAGTCTCGTCGGATGTGTTCACCAAATTT | |
| | AAGAACGCAAATGGGGAATTCGAATCCGAT | |
| | GACGCAAGAAGTTGGTGGTCCCTGTATGAG | |
| | GCAACCCACCCAAGAACTCCAGAAGAATCC | |
| | ATCTTGGAAGAAGCATTAAAAATCAGTAGA | |
| | GAAAAGCTAGAGGCTCTGTTACCCCAACTT | |
| | TCTTTTCCGTTGTCGGATTGTGTGCGTCAT | |
| | GCCTTACACATACCTTATTACAGGAATGTT | |
| | CCAAGATTAGTAGCTCGTCAGTATATTCCT | |
| | CTATACGATGCTGAAGCCACTAAGATTGAA | |
| | AGCCTTGCTAGGTTTGCTAAAATAGATTTC | |
| | AACATGTTACAGGCCCTTCATCAATCTGAA | |
| | CTTAAAGAAGTGTCAAGATGGTGGAAGGAT | |
| | TTTGACTTCGCAACTAAGTTACCATACGCA | |
| | CGTGATAGAATTTCAGAGGGGTACTACTGG | |
| | ATTATGGGTGCGCACCACGAACCTAAGTTT | |
| | TCATTATCAAGAAAGTTTTTAACTAAAATC | |
| | ATAGGGATAACATCACTTATAGACGATACG | |
| | TATGATGTTTACGGTAACTTAGAAGAAGTC | |
| | ACCAAATTCACTGAGGCCGTGGAAAGATGG | |
| | GATATGGAAGCTTTGAACGACATCCCTCAG | |
| | TACATGCAAGTTATTTACACGGGGTTGTTA | |
| | GGAATGTATGAGGAATTCAAGGACGATTTG | |
| | ATAAATGCAGGAGGCAGAGATTTCGCACTT | |
| | TACTATGCAATAGAAGTAATGAAGGAAATA | |
| | GTAAGGAGTTATCAAAAAGAAGCAGAGTTT | |
| | TTCCATACCGGTTATACACCCGGTTTTGAT | |
| | GAATACATGGAAAACTCTATTATTAGTGGA | |
| | GGCTACAAAATGTTTATTATACTGGAAGTG | |
| | ATTACGAGAGGGGAGTATTCTCTTAAAGAA | |
| | ACATTAGACTGGGCTCTAACAGTTCCTGAG | |
| | ATGGTTAAAAGTTCATCGTTAATTGCACGT | |
| | TTTGTTGACGACATCCAAACATATAAAGCT | |
| | GAAGAAGAGAGAGGAGAGGATGTTTCCGCA | |
| | GTCAGATGCTATATGAAAGAATATGGGGTG | |
| | TCCGAAGAGGAAGCTTGTAAAAAGATGAGG | |
| | GAAATGATAGAAATAGAATGGAAGAAACTA | |
| | AATAAAGAAACTCAAGAAACTGACGAAGTG | |
| | ACAGCTCCAGCAGTTCTACCATCCTTAAAC | |
| | TTTACCAGAGTTCTTGAAGTTATGTACGAT | |
| | GAGGGTGATGGTTATTCCAACTCACAAGGC | |
| | GTCACAAAAGATCGTATTACTGCTCTATTA | |
| | CAAAATCCAATTGATCTTTGA | |
| 2 | MSVSPRLKRVSSGKFAFTSDAVTRRDANYH | Polypeptide |
| | PTVWGDYFLNHSSKFLETNENIREEHERLG | SchTPS1 |
| | LEARSLLVDEKMDHPNKIQLIDDIIRLGVD | Sequence |
| | YHFESEINAELKKLHDDHDHLDFDLRTTST | |
| | LFRLLRAHGFQVSSDVFTKFKNANGEFESD | |
| | DARSWWSLYEATHPRTPEESILEEALKISR | |
| | EKLEALLPQLSFPLSDCVRHALHIPYYRNV | |
| | PRLVARQYIPLYDAEATKIESLARFAKIDE | |
| | NMLQALHQSELKEVSRWWKDFDFATKLPYA | |
| | RDRISEGYYWIMGAHHEPKFSLSRKFLTKI | |
| | IGITSLIDDTYDVYGNLEEVTKFTEAVERW | |
| | DMEALNDIPQYMQVIYTGLLGMYEEFKDDL | |
| | INAGGRDFALYYAIEVMKEIVRSYQKEAEF | |
| | FHTGYTPGFDEYMENSIISGGYKMFIILEV | |
| | ITRGEYSLKETLDWALTVPEMVKSSSLIAR | |
| | FVDDIQTYKAEEERGEDVSAVRCYMKEYGV | |
| | SEEEACKKMREMIEIEWKKLNKETQETDEV | |
| | TAPAVLPSLNFTRVLEVMYDEGDGYSNSQG | |
| | VTKDRITALLQNPIDL | |
| 3 | ATGTCCTCGACTAAAATAGAATCTGCCTCA | Sequence |
| | GATGACGCATCCAGAAGAGACGCGAACTTT | SchTPS3 DNA |
| | CATCCTACGGTGTGGGGAGACTTCTTTCTG | |
| | AATTTGAGCAGCAACTTCTTGGAAAATAAT | |
| | AAAGACGTTCAAGAAAAACACGAGAAATTA | |
| | AGAGAAGAAGTTAAAAAACTGGTTGTAGAT | |
| | CACCACCATCACGATAGTGCTGACGATATT | |
| | CCAAGTAAAATCCAATTGATTGATGATATT | |
| | ATAAGGTTAGGCGTTGAATATCATTTCGAA | |
| | GAAGAGATTAAAGCGGAATTGGAAAAACTA | |
| | CATGATAACCAACTTCATCTTAATTTTGAT | |
| | CTAGCTACTACATCTACTTGGTTGAGACTA | |
| | TTAAGAACGCATGGTTTTAGTGTAAGTTCT | |
| | GATGTTTTTAAGAGATTTAAAAACGCAAAC | |
| | GGCGAATTTGAGTCAGAAGATGCAAGAGGT | |
| | TGGTGGTGCCTATATGAGGCCACTCATTTG | |
| | CGTGTGCCTGATGACAAGATTTTAGAAGAG | |
| | GCATTACAAGTCTCGAGGAAGAAGCTTGAG | |
| | GCTCTATTAACCGAATTAACTTTTCCTCTG | |
| | AAAGAATGCGTTAGAAACGCTCTTCATATT | |
| | CCCTACTATCGTAGCGTTCCTCGTTTAGAA | |
| | GCGAGACAGTATATTCCATCTTACGACGCG | |
| | CAGCCAACCAAAATAGAAAGTTTGAGTTTA | |
| | TTCGCTAAAATTGACTTCAATATGTTGCAG | |
| | GCCTTGCATCAAATTGAGTTAAGAGAAGTT | |
| | TCAAGATGGTGGAAAGACTTCGACTTTAGT | |
| | AACAAGTTGCCATATGCAAGAGATAGAATT | |
| | GCCGAAGGTTATTACTGGATCATGGGCGCA | |
| | CACCATGAACCTAAGTTTAGTTTAAGTAGA | |
| | AAGTTTTTGACTAAGGTAATTGGGATTACA | |
| | TCTTTGATTGACGATACTTACGATGTCTAC | |
| | GGTTCATTGGAAGAGGTTACTTTATTTACA | |
| | AAAGCGGTAGAAAGATGGGATATAGAAGCA | |
| | ATAAACGATATACCAGAATACATGAGGGTT | |
| | ATTTACATAGGTCTTCTTGGTATGTTCGAG | |
| | GAGTACAAGGAAAATCTTATAAATGCAGGG | |
| | GGAAGAGAATATTGTTTGTATTACGCTATT | |
| | GAAGTTATGAAAGAGATAATTCGTTGCTAT | |
| | CAGAAGGAGGCCGAATTCTTCCATAAAGGG | |
| | TACGTCCCTGGGTATGAAGAGTACATGGAA | |
| | AACTCTGTGATATCTGGTGGCTACAAAATG | |
| | TTTATAATCTTGGAAGTTATTACGAGAGGC | |
| | GAATTTGGTGTAAAAGAAACGCTTGACTGG | |
| | GCGATGGGTGTTCCTGAAATGGTAAAATCG | |
| | TCTAGTCTAATTGCCAGATTCGTCGATGAT | |
| | GTTCAAACTTATAAAGCCGAAGAAGAGAGA | |
| | GGAGAAGTGGTAAGCGCGATTAGGTGCTAT | |
| | ATGAAAGATTATGGTGTTTCTGAAGAGGAA | |
| | GCGTGCGGCAAAATGAGAGAAATGATTGAA | |
| | ATGGAGTGGCAAAAGCTAAATAAGGAGACC | |
| | CAAGAAAGGAGGAACGAGGTATCCGCTCCA | |
| | GTAGTTATCCCCAGTTTAAATTTCACGAGA | |
| | GTACTTGAAGTTATGTATGACCAAGGAGAC | |
| | GGGTACTCTGACTCTCAAGGCGTTACTAAA | |
| | GACAGGATTACAGCCTTGCTTAGACATCCT | |
| | ATCGAAATATGA | |
| 4 | MSSTKIESASDDASRRDANFHPTVWGDFFL | SchTPS3 |
| | NLSSNFLENNKDVQEKHEKLREEVKKLVVD | polypeptide |
| | HHHHDSADDIPSKIQLIDDIIRLGVEYHFE | Sequence |
| | EEIKAELEKLHDNQLHLNFDLATTSTWLRL | |
| | LRTHGFSVSSDVFKRFKNANGEFESEDARG | |
| | WWCLYEATHLRVPDDKILEEALQVSRKKLE | |
| | ALLTELTFPLKECVRNALHIPYYRSVPRLE | |
| | ARQYIPSYDAQPTKIESLSLFAKIDFNMLQ | |
| | ALHQIELREVSRWWKDFDFSNKLPYARDRI | |
| | AEGYYWIMGAHHEPKFSLSRKFLTKVIGIT | |
| | SLIDDTYDVYGSLEEVTLFTKAVERWDIEA | |
| | INDIPEYMRVIYIGLLGMFEEYKENLINAG | |
| | GREYCLYYAIEVMKEIIRCYQKEAEFFHKG | |
| | YVPGYEEYMENSVISGGYKMFIILEVITRG | |
| | EFGVKETLDWAMGVPEMVKSSSLIARFVDD | |
| | VQTYKAEEERGEVVSAIRCYMKDYGVSEEE | |
| | ACGKMREMIEMEWQKLNKETQERRNEVSAP | |
| | VVIPSLNFTRVLEVMYDQGDGYSDSQGVTK | |
| | DRITALLRHPIEI | |
| 5 | ATGGTATCCTCTGGAAAGTTCGCCTTTACA | SchTPS1- |
| | TCAGATGCAGTTACTAGAAGAGATGCTAAT | truncation |
| | TACCATCCAACCGTCTGGGGTGATTATTTT | DNA Sequence |
| | CTAAACCATTCTAGTAAATTCTTAGAAACT | |
| | AATGAAAACATTAGAGAAGAACACGAAAGG | |
| | TTGGGTCTTGAAGCAAGATCCCTGTTGGTA | |
| | GATGAGAAAATGGATCACCCAAACAAAATT | |
| | CAATTGATTGATGACATTATTAGGCTTGGG | |
| | GTCGATTACCATTTTGAATCGGAAATCAAC | |
| | GCTGAACTAAAGAAACTGCATGATGACCAC | |
| | GACCACTTAGACTTCGATTTAAGAACCACT | |
| | TCCACTTTGTTCAGATTACTAAGAGCACAT | |
| | GGTTTTCAAGTCTCGTCGGATGTGTTCACC | |
| | AAATTTAAGAACGCAAATGGGGAATTCGAA | |
| | TCCGATGACGCAAGAAGTTGGTGGTCCCTG | |
| | TATGAGGCAACCCACCCAAGAACTCCAGAA | |
| | GAATCCATCTTGGAAGAAGCATTAAAAATC | |
| | AGTAGAGAAAAGCTAGAGGCTCTGTTACCC | |
| | CAACTTTCTTTTCCGTTGTCGGATTGTGTG | |
| | CGTCATGCCTTACACATACCTTATTACAGG | |
| | AATGTTCCAAGATTAGTAGCTCGTCAGTAT | |
| | ATTCCTCTATACGATGCTGAAGCCACTAAG | |
| | ATTGAAAGCCTTGCTAGGTTTGCTAAAATA | |
| | GATTTCAACATGTTACAGGCCCTTCATCAA | |
| | TCTGAACTTAAAGAAGTGTCAAGATGGTGG | |
| | AAGGATTTTGACTTCGCAACTAAGTTACCA | |
| | TACGCACGTGATAGAATTTCAGAGGGGTAC | |
| | TACTGGATTATGGGTGCGCACCACGAACCT | |
| | AAGTTTTCATTATCAAGAAAGTTTTTAACT | |
| | AAAATCATAGGGATAACATCACTTATAGAC | |
| | GATACGTATGATGTTTACGGTAACTTAGAA | |
| | GAAGTCACCAAATTCACTGAGGCCGTGGAA | |
| | AGATGGGATATGGAAGCTTTGAACGACATC | |
| | CCTCAGTACATGCAAGTTATTTACACGGGG | |
| | TTGTTAGGAATGTATGAGGAATTCAAGGAC | |
| | GATTTGATAAATGCAGGAGGCAGAGATTTC | |
| | GCACTTTACTATGCAATAGAAGTAATGAAG | |
| | GAAATAGTAAGGAGTTATCAAAAAGAAGCA | |
| | GAGTTTTTCCATACCGGTTATACACCCGGT | |
| | TTTGATGAATACATGGAAAACTCTATTATT | |
| | AGTGGAGGCTACAAAATGTTTATTATACTG | |
| | GAAGTGATTACGAGAGGGGAGTATTCTCTT | |
| | AAAGAAACATTAGACTGGGCTCTAACAGTT | |
| | CCTGAGATGGTTAAAAGTTCATCGTTAATT | |
| | GCACGTTTTGTTGACGACATCCAAACATAT | |
| | AAAGCTGAAGAAGAGAGAGGAGAGGATGTT | |
| | TCCGCAGTCAGATGCTATATGAAAGAATAT | |
| | GGGGTGTCCGAAGAGGAAGCTTGTAAAAAG | |
| | ATGAGGGAAATGATAGAAATAGAATGGAAG | |
| | AAACTAAATAAAGAAACTCAAGAAACTGAC | |
| | GAAGTGACAGCTCCAGCAGTTCTACCATCC | |
| | TTAAACTTTACCAGAGTTCTTGAAGTTATG | |
| | TACGATGAGGGTGATGGTTATTCCAACTCA | |
| | CAAGGCGTCACAAAAGATCGTATTACTGCT | |
| | CTATTACAAAATCCAATTGATCTTTGA | |
| 6 | MVSSGKFAFTSDAVTRRDANYHPTVWGDYF | SchTPS1- |
| | LNHSSKFLETNENIREEHERLGLEARSLLV | truncation |
| | DEKMDHPNKIQLIDDIIRLGVDYHFESEIN | Polypeptide |
| | AELKKLHDDHDHLDFDLRTTSTLFRLLRAH | Sequence |
| | GFQVSSDVFTKFKNANGEFESDDARSWWSL | |
| | YEATHPRTPEESILEEALKISREKLEALLP | |
| | QLSFPLSDCVRHALHIPYYRNVPRLVARQY | |
| | IPLYDAEATKIESLARFAKIDENMLQALHQ | |
| | SELKEVSRWWKDFDFATKLPYARDRISEGY | |
| | YWIMGAHHEPKFSLSRKFLTKIIGITSLID | |
| | DTYDVYGNLEEVTKFTEAVERWDMEALNDI | |
| | PQYMQVIYTGLLGMYEEFKDDLINAGGRDF | |
| | ALYYAIEVMKEIVRSYQKEAEFFHTGYTPG | |
| | FDEYMENSIISGGYKMFIILEVITRGEYSL | |
| | KETLDWALTVPEMVKSSSLIARFVDDIQTY | |
| | KAEEERGEDVSAVRCYMKEYGVSEEEACKK | |
| | MREMIEIEWKKLNKETQETDEVTAPAVLPS | |
| | LNFTRVLEVMYDEGDGYSNSQGVTKDRITA | |
| | LLQNPIDL | |
| 7 | ATGTCCTCGACTAAAATAGAATCTGCCTCA | SchTPS3- |
| | GATGACGCATCCAGAAGAGACGCGAACTTT | truncation |
| | CATCCTACGGTGTGGGGAGACTTCTTTCTG | DNA |
| | AATTTGAGCAGCAACTTCTTGGAAAATAAT | Sequence |
| | AAAGACGTTCAAGAAAAACACGAGAAATTA | |
| | AGAGAAGAAGTTAAAAAACTGGTTGTAGAT | |
| | AGTGCTGACGATATTCCAAGTAAAATCCAA | |
| | TTGATTGATGATATTATAAGGTTAGGCGTT | |
| | GAATATCATTTCGAAGAAGAGATTAAAGCG | |
| | GAATTGGAAAAACTACATGATAACCAACTT | |
| | CATCTTAATTTTGATCTAGCTACTACATCT | |
| | ACTTGGTTGAGACTATTAAGAACGCATGGT | |
| | TTTAGTGTAAGTTCTGATGTTTTTAAGAGA | |
| | TTTAAAAACGCAAACGGCGAATTTGAGTCA | |
| | GAAGATGCAAGAGGTTGGTGGTGCCTATAT | |
| | GAGGCCACTCATTTGCGTGTGCCTGATGAC | |
| | AAGATTTTAGAAGAGGCATTACAAGTCTCG | |
| | AGGAAGAAGCTTGAGGCTCTATTAACCGAA | |
| | TTAACTTTTCCTCTGAAAGAATGCGTTAGA | |
| | AACGCTCTTCATATTCCCTACTATCGTAGC | |
| | GTTCCTCGTTTAGAAGCGAGACAGTATATT | |
| | CCATCTTACGACGCGCAGCCAACCAAAATA | |
| | GAAAGTTTGAGTTTATTCGCTAAAATTGAC | |
| | TTCAATATGTTGCAGGCCTTGCATCAAATT | |
| | GAGTTAAGAGAAGTTTCAAGATGGTGGAAA | |
| | GACTTCGACTTTAGTAACAAGTTGCCATAT | |
| | GCAAGAGATAGAATTGCCGAAGGTTATTAC | |
| | TGGATCATGGGCGCACACCATGAACCTAAG | |
| | TTTAGTTTAAGTAGAAAGTTTTTGACTAAG | |
| | GTAATTGGGATTACATCTTTGATTGACGAT | |
| | ACTTACGATGTCTACGGTTCATTGGAAGAG | |
| | GTTACTTTATTTACAAAAGCGGTAGAAAGA | |
| | TGGGATATAGAAGCAATAAACGATATACCA | |
| | GAATACATGAGGGTTATTTACATAGGTCTT | |
| | CTTGGTATGTTCGAGGAGTACAAGGAAAAT | |
| | CTTATAAATGCAGGGGGAAGAGAATATTGT | |
| | TTGTATTACGCTATTGAAGTTATGAAAGAG | |
| | ATAATTCGTTGCTATCAGAAGGAGGCCGAA | |
| | TTCTTCCATAAAGGGTACGTCCCTGGGTAT | |
| | GAAGAGTACATGGAAAACTCTGTGATATCT | |
| | GGTGGCTACAAAATGTTTATAATCTTGGAA | |
| | GTTATTACGAGAGGCGAATTTGGTGTAAAA | |
| | GAAACGCTTGACTGGGCGATGGGTGTTCCT | |
| | GAAATGGTAAAATCGTCTAGTCTAATTGCC | |
| | AGATTCGTCGATGATGTTCAAACTTATAAA | |
| | GCCGAAGAAGAGAGAGGAGAAGTGGTAAGC | |
| | GCGATTAGGTGCTATATGAAAGATTATGGT | |
| | GTTTCTGAAGAGGAAGCGTGCGGCAAAATG | |
| | AGAGAAATGATTGAAATGGAGTGGCAAAAG | |
| | CTAAATAAGGAGACCCAAGAAAGGAGGAAC | |
| | GAGGTATCCGCTCCAGTAGTTATCCCCAGT | |
| | TTAAATTTCACGAGAGTACTTGAAGTTATG | |
| | TATGACCAAGGAGACGGGTACTCTGACTCT | |
| | CAAGGCGTTACTAAAGACAGGATTACAGCC | |
| | TTGCTTAGACATCCTATCGAAATATGA | |
| 8 | MSSTKIESASDDASRRDANFHPTVWGDFFL | SchTPS3- |
| | NLSSNFLENNKDVQEKHEKLREEVKKLVVD | truncation |
| | SADDIPSKIQLIDDIIRLGVEYHFEEEIKA | Polypeptide |
| | ELEKLHDNQLHLNFDLATTSTWLRLLRTHG | Sequence |
| | FSVSSDVFKRFKNANGEFESEDARGWWCLY | |
| | EATHLRVPDDKILEEALQVSRKKLEALLTE | |
| | LTFPLKECVRNALHIPYYRSVPRLEARQYI | |
| | PSYDAQPTKIESLSLFAKIDFNMLQALHQI | |
| | ELREVSRWWKDFDFSNKLPYARDRIAEGYY | |
| | WIMGAHHEPKFSLSRKFLTKVIGITSLIDD | |
| | TYDVYGSLEEVTLFTKAVERWDIEAINDIP | |
| | EYMRVIYIGLLGMFEEYKENLINAGGREYC | |
| | LYYAIEVMKEIIRCYQKEAEFFHKGYVPGY | |
| | EEYMENSVISGGYKMFIILEVITRGEFGVK | |
| | ETLDWAMGVPEMVKSSSLIARFVDDVQTYK | |
| | AEEERGEVVSAIRCYMKDYGVSEEEACGKM | |
| | REMIEMEWQKLNKETQERRNEVSAPVVIPS | |
| | LNFTRVLEVMYDQGDGYSDSQGVTKDRITA | |
| | LLRHPIEI | |

### REFERENCES

1 An et al. Characterization of Guaiene Synthases from Stellera chamaejasme L. Flowers and Their Application in De novo Production of (−)-Rotundone in Yeast. J. Agric. Food Chem. 2020, 68, 10, 3214-321920.
2. Gietz et al., Yeast transformation by the LiAc/SS Carrier DNA/PEG method. Methods Mol Biol. 2006; 313:107-20
3. Kumeta and Ito. Characterization of δ-Guaiene Synthases from Cultured Cells of Aquilaria, Responsible for the Formation of the Sesquiterpenes in Agarwood. Plant Physiology, December 2010, Vol. 154, pp. 1998-2007.
4. Drew et al. Two key polymorphisms in a newly discovered allele of the Vitis vinifera TPS24 gene are responsible for the production of the rotundone precursor α-guaiene. Journal of Experimental Botany, 2016. Vol. 67, No. 3 pp. 799-808. Rost. Twilight zone of protein sequence alignments. Protein Engineering, Design and Selection, 1999. Vol. 12, No. 2 pp. 85-94.
5. Wang Gietz et al., Yeast transformation by the LiAc/SS Carrier DNA/PEG method. Methods Mol Biol. 2006; 313:107-20. A benchmark study of sequence alignment methods for protein clustering. BMC Bioinformatics, 2018. Vol. 19, Supplement 19 pp. 95-104.
6 Wiltgen. Algorithms for Structure Comparison and Analysis: Homology Modelling of Proteins. In S. Ranganathan, M. Gribskov, K. Nakai, C. Schönbach (Ed.), Encyclopedia of Bioinformatics and Computational Biology, 2019. Academic Press.

What is claimed is:

1. An engineered polypeptide encoding a guaiene synthase, wherein the engineered polypeptide comprises an amino acid sequence at least about 85% identical to SEQ ID NO:4 and wherein the amino acid sequence comprises a deletion of amino acids corresponding to positions 60-64 of SEQ ID NO:4.

2-40. (canceled)

41. The engineered polypeptide of claim 1, wherein the engineered polypeptide comprises an amino acid sequence at least about 90% identical to SEQ ID NO:8.

42. The engineered polypeptide of claim 1, wherein the engineered polypeptide comprises an amino acid sequence at least about 95% identical to SEQ ID NO:8.

43. The engineered polypeptide of claim 1, wherein the engineered polypeptide comprises an amino acid sequence at least about 98% identical to SEQ ID NO:8.

44. The engineered polypeptide of claim 1, wherein the engineered polypeptide comprises an amino acid sequence that is SEQ ID NO:8.

45. A cell expressing the engineered polypeptide of claim 1.

46. The cell of claim 45, wherein the engineered polypeptide comprises an amino acid sequence at least about 90% identical to SEQ ID NO:8.

47. The cell of claim 45, wherein the engineered polypeptide comprises an amino acid sequence at least about 95% identical to SEQ ID NO:8.

48. The cell of claim 45, wherein the engineered polypeptide comprises an amino acid sequence at least about 98% identical to SEQ ID NO:8.

49. The cell of claim 45, wherein the engineered polypeptide comprises an amino acid sequence that is SEQ ID NO:8.

50. The cell of claim 45, wherein the cell is a microbial cell.

51. The cell of claim 50, wherein the microbial cell is yeast or E. coli.

52. The cell of claim 51, wherein the microbial cell is yeast.

53. The cell of claim 52, wherein the yeast is Saccharomyces cerevisiae.

54. The cell of claim 45, capable of producing total guaiene products comprising alpha-guaiene, wherein the percentage of the alpha-guaiene is at least greater than about 15% of the total guaiene products.

55. The cell of claim 54, wherein the percentage of alpha-guaiene is about 30% to about 90% of the total guaiene products.

56. A nucleic acid molecule encoding the engineered polypeptide of claim 1.

57. The nucleic acid molecule of claim 56, comprising a nucleic acid sequence of SEQ ID NO: 7.

58. A method for producing guaiene, the method comprising:
growing the cell of claim 45 in a culture, and
collecting guaiene from the culture.


SEQUENCE LISTING (8 sequences)

SEQ ID NO: 1 - DNA, 1671 bp, synthetic construct
  source 1..1671: mol_type = other DNA
  misc_feature 1..1671: note = SchTPS1
atgagtgtatctccgaggttaaagagagtatcctctggaaagttcgcctttacatcagatgcagttactagaagagatgctaattaccatccaaccgtctggggtgattattttctaaaccattctagtaaattcttagaaactaatgaaaacattagagaagaacacgaaaggttgggtcttgaagcaagatccctgttggtagatgagaaaatggatcacccaaacaaaattcaattgattgatgacattattaggcttggggtcgattaccattttgaatcggaaatcaacgctgaactaaagaaactgcatgatgaccacgaccacttagacttcgatttaagaaccacttccactttgttcagattactaagagcacatggttttcaagtctcgtcggatgtgttcaccaaatttaagaacgcaaatggggaattcgaatccgatgacgcaagaagttggtggtccctgtatgaggcaacccacccaagaactccagaagaatccatcttggaagaagcattaaaaatcagtagagaaaagctagaggctctgttaccccaactttcttttccgttgtcggattgtgtgcgtcatgccttacacataccttattacaggaatgttccaagattagtagctcgtcagtatattcctctatacgatgctgaagccactaagattgaaagccttgctaggtttgctaaaatagatttcaacatgttacaggcccttcatcaatctgaacttaaagaagtgtcaagatggtggaaggattttgacttcgcaactaagttaccatacgcacgtgatagaatttcagaggggtactactggattatgggtgcgcaccacgaacctaagttttcattatcaagaaagtttttaactaaaatcatagggataacatcacttatagacgatacgtatgatgtttacggtaacttagaagaagtcaccaaattcactgaggccgtggaaagatgggatatggaagctttgaacgacatccctcagtacatgcaagttatttacacggggttgttaggaatgtatgaggaattcaaggacgatttgataaatgcaggaggcagagatttcgcactttactatgcaatagaagtaatgaaggaaatagtaaggagttatcaaaaagaagcagagtttttccataccggttatacacccggttttgatgaatacatggaaaactctattattagtggaggctacaaaatgtttattatactggaagtgattacgagaggggagtattctcttaaagaaacattagactgggctctaacagttcctgagatggttaaaagttcatcgttaattgcacgttttgttgacgacatccaaacatataaagctgaagaagagagaggagaggatgtttccgcagtcagatgctatatgaaagaatatggggtgtccgaagaggaagcttgtaaaaagatgagggaaatgatagaaatagaatggaagaaactaaataaagaaactcaagaaactgacgaagtgacagctccagcagttctaccatccttaaactttaccagagttcttgaagttatgtacgatgagggtgatggttattccaactcacaaggcgtcacaaaagatcgtattactgctctattacaaaatccaattgatctttga

SEQ ID NO: 2 - protein, 556 aa, synthetic construct
  REGION 1..556: note = SchTPS1
MSVSPRLKRVSSGKFAFTSDAVTRRDANYHPTVWGDYFLNHSSKFLETNENIREEHERLGLEARSLLVDEKMDHPNKIQLIDDIIRLGVDYHFESEINAELKKLHDDHDHLDFDLRTTSTLFRLLRAHGFQVSSDVFTKFKNANGEFESDDARSWWSLYEATHPRTPEESILEEALKISREKLEALLPQLSFPLSDCVRHALHIPYYRNVPRLVARQYIPLYDAEATKIESLARFAKIDFNMLQALHQSELKEVSRWWKDFDFATKLPYARDRISEGYYWIMGAHHEPKFSLSRKFLTKIIGITSLIDDTYDVYGNLEEVTKFTEAVERWDMEALNDIPQYMQVIYTGLLGMYEEFKDDLINAGGRDFALYYAIEVMKEIVRSYQKEAEFFHTGYTPGFDEYMENSIISGGYKMFIILEVITRGEYSLKETLDWALTVPEMVKSSSLIARFVDDIQTYKAEEERGEDVSAVRCYMKEYGVSEEEACKKMREMIEIEWKKLNKETQETDEVTAPAVLPSLNFTRVLEVMYDEGDGYSNSQGVTKDRITALLQNPIDL

SEQ ID NO: 3 - DNA, 1662 bp, synthetic construct
  source 1..1662: mol_type = other DNA
  misc_feature 1..1662: note = SchTPS3
atgtcctcgactaaaatagaatctgcctcagatgacgcatccagaagagacgcgaactttcatcctacggtgtggggagacttctttctgaatttgagcagcaacttcttggaaaataataaagacgttcaagaaaaacacgagaaattaagagaagaagttaaaaaactggttgtagatcaccaccatcacgatagtgctgacgatattccaagtaaaatccaattgattgatgatattataaggttaggcgttgaatatcatttcgaagaagagattaaagcggaattggaaaaactacatgataaccaacttcatcttaattttgatctagctactacatctacttggttgagactattaagaacgcatggttttagtgtaagttctgatgtttttaagagatttaaaaacgcaaacggcgaatttgagtcagaagatgcaagaggttggtggtgcctatatgaggccactcatttgcgtgtgcctgatgacaagattttagaagaggcattacaagtctcgaggaagaagcttgaggctctattaaccgaattaacttttcctctgaaagaatgcgttagaaacgctcttcatattccctactatcgtagcgttcctcgtttagaagcgagacagtatattccatcttacgacgcgcagccaaccaaaatagaaagtttgagtttattcgctaaaattgacttcaatatgttgcaggccttgcatcaaattgagttaagagaagtttcaagatggtggaaagacttcgactttagtaacaagttgccatatgcaagagatagaattgccgaaggttattactggatcatgggcgcacaccatgaacctaagtttagtttaagtagaaagtttttgactaaggtaattgggattacatctttgattgacgatacttacgatgtctacggttcattggaagaggttactttatttacaaaagcggtagaaagatgggatatagaagcaataaacgatataccagaatacatgagggttatttacataggtcttcttggtatgttcgaggagtacaaggaaaatcttataaatgcagggggaagagaatattgtttgtattacgctattgaagttatgaaagagataattcgttgctatcagaaggaggccgaattcttccataaagggtacgtccctgggtatgaagagtacatggaaaactctgtgatatctggtggctacaaaatgtttataatcttggaagttattacgagaggcgaatttggtgtaaaagaaacgcttgactgggcgatgggtgttcctgaaatggtaaaatcgtctagtctaattgccagattcgtcgatgatgttcaaacttataaagccgaagaagagagaggagaagtggtaagcgcgattaggtgctatatgaaagattatggtgtttctgaagaggaagcgtgcggcaaaatgagagaaatgattgaaatggagtggcaaaagctaaataaggagacccaagaaaggaggaacgaggtatccgctccagtagttatccccagtttaaatttcacgagagtacttgaagttatgtatgaccaaggagacgggtactctgactctcaaggcgttactaaagacaggattacagccttgcttagacatcctatcgaaatatga

SEQ ID NO: 4 - protein, 553 aa, synthetic construct
  REGION 1..553: note = SchTPS3
MSSTKIESASDDASRRDANFHPTVWGDFFLNLSSNFLENNKDVQEKHEKLREEVKKLVVDHHHHDSADDIPSKIQLIDDIIRLGVEYHFEEEIKAELEKLHDNQLHLNFDLATTSTWLRLLRTHGFSVSSDVFKRFKNANGEFESEDARGWWCLYEATHLRVPDDKILEEALQVSRKKLEALLTELTFPLKECVRNALHIPYYRSVPRLEARQYIPSYDAQPTKIESLSLFAKIDFNMLQALHQIELREVSRWWKDFDFSNKLPYARDRIAEGYYWIMGAHHEPKFSLSRKFLTKVIGITSLIDDTYDVYGSLEEVTLFTKAVERWDIEAINDIPEYMRVIYIGLLGMFEEYKENLINAGGREYCLYYAIEVMKEIIRCYQKEAEFFHKGYVPGYEEYMENSVISGGYKMFIILEVITRGEFGVKETLDWAMGVPEMVKSSSLIARFVDDVQTYKAEEERGEVVSAIRCYMKDYGVSEEEACGKMREMIEMEWQKLNKETQERRNEVSAPVVIPSLNFTRVLEVMYDQGDGYSDSQGVTKDRITALLRHPIEI

SEQ ID NO: 5 - DNA, 1647 bp, synthetic construct
  source 1..1647: mol_type = other DNA
  misc_feature 1..1647: note = SchTPS1 - truncation
atggtatcctctggaaagttcgcctttacatcagatgcagttactagaagagatgctaattaccatccaaccgtctggggtgattattttctaaaccattctagtaaattcttagaaactaatgaaaacattagagaagaacacgaaaggttgggtcttgaagcaagatccctgttggtagatgagaaaatggatcacccaaacaaaattcaattgattgatgacattattaggcttggggtcgattaccattttgaatcggaaatcaacgctgaactaaagaaactgcatgatgaccacgaccacttagacttcgatttaagaaccacttccactttgttcagattactaagagcacatggttttcaagtctcgtcggatgtgttcaccaaatttaagaacgcaaatggggaattcgaatccgatgacgcaagaagttggtggtccctgtatgaggcaacccacccaagaactccagaagaatccatcttggaagaagcattaaaaatcagtagagaaaagctagaggctctgttaccccaactttcttttccgttgtcggattgtgtgcgtcatgccttacacataccttattacaggaatgttccaagattagtagctcgtcagtatattcctctatacgatgctgaagccactaagattgaaagccttgctaggtttgctaaaatagatttcaacatgttacaggcccttcatcaatctgaacttaaagaagtgtcaagatggtggaaggattttgacttcgcaactaagttaccatacgcacgtgatagaatttcagaggggtactactggattatgggtgcgcaccacgaacctaagttttcattatcaagaaagtttttaactaaaatcatagggataacatcacttatagacgatacgtatgatgtttacggtaacttagaagaagtcaccaaattcactgaggccgtggaaagatgggatatggaagctttgaacgacatccctcagtacatgcaagttatttacacggggttgttaggaatgtatgaggaattcaaggacgatttgataaatgcaggaggcagagatttcgcactttactatgcaatagaagtaatgaaggaaatagtaaggagttatcaaaaagaagcagagtttttccataccggttatacacccggttttgatgaatacatggaaaactctattattagtggaggctacaaaatgtttattatactggaagtgattacgagaggggagtattctcttaaagaaacattagactgggctctaacagttcctgagatggttaaaagttcatcgttaattgcacgttttgttgacgacatccaaacatataaagctgaagaagagagaggagaggatgtttccgcagtcagatgctatatgaaagaatatggggtgtccgaagaggaagcttgtaaaaagatgagggaaatgatagaaatagaatggaagaaactaaataaagaaactcaagaaactgacgaagtgacagctccagcagttctaccatccttaaactttaccagagttcttgaagttatgtacgatgagggtgatggttattccaactcacaaggcgtcacaaaagatcgtattactgctctattacaaaatccaattgatctttga

SEQ ID NO: 6 - protein, 548 aa, synthetic construct
  REGION 1..548: note = SchTPS1 - truncation
MVSSGKFAFTSDAVTRRDANYHPTVWGDYFLNHSSKFLETNENIREEHERLGLEARSLLVDEKMDHPNKIQLIDDIIRLGVDYHFESEINAELKKLHDDHDHLDFDLRTTSTLFRLLRAHGFQVSSDVFTKFKNANGEFESDDARSWWSLYEATHPRTPEESILEEALKISREKLEALLPQLSFPLSDCVRHALHIPYYRNVPRLVARQYIPLYDAEATKIESLARFAKIDFNMLQALHQSELKEVSRWWKDFDFATKLPYARDRISEGYYWIMGAHHEPKFSLSRKFLTKIIGITSLIDDTYDVYGNLEEVTKFTEAVERWDMEALNDIPQYMQVIYTGLLGMYEEFKDDLINAGGRDFALYYAIEVMKEIVRSYQKEAEFFHTGYTPGFDEYMENSIISGGYKMFIILEVITRGEYSLKETLDWALTVPEMVKSSSLIARFVDDIQTYKAEEERGEDVSAVRCYMKEYGVSEEEACKKMREMIEIEWKKLNKETQETDEVTAPAVLPSLNFTRVLEVMYDEGDGYSNSQGVTKDRITALLQNPIDL

SEQ ID NO: 7 - DNA, 1647 bp, synthetic construct
  source 1..1647: mol_type = other DNA
  misc_feature 1..1647: note = SchTPS3 - truncation
atgtcctcgactaaaatagaatctgcctcagatgacgcatccagaagagacgcgaactttcatcctacggtgtggggagacttctttctgaatttgagcagcaacttcttggaaaataataaagacgttcaagaaaaacacgagaaattaagagaagaagttaaaaaactggttgtagatagtgctgacgatattccaagtaaaatccaattgattgatgatattataaggttaggcgttgaatatcatttcgaagaagagattaaagcggaattggaaaaactacatgataaccaacttcatcttaattttgatctagctactacatctacttggttgagactattaagaacgcatggttttagtgtaagttctgatgtttttaagagatttaaaaacgcaaacggcgaatttgagtcagaagatgcaagaggttggtggtgcctatatgaggccactcatttgcgtgtgcctgatgacaagattttagaagaggcattacaagtctcgaggaagaagcttgaggctctattaaccgaattaacttttcctctgaaagaatgcgttagaaacgctcttcatattccctactatcgtagcgttcctcgtttagaagcgagacagtatattccatcttacgacgcgcagccaaccaaaatagaaagtttgagtttattcgctaaaattgacttcaatatgttgcaggccttgcatcaaattgagttaagagaagtttcaagatggtggaaagacttcgactttagtaacaagttgccatatgcaagagatagaattgccgaaggttattactggatcatgggcgcacaccatgaacctaagtttagtttaagtagaaagtttttgactaaggtaattgggattacatctttgattgacgatacttacgatgtctacggttcattggaagaggttactttatttacaaaagcggtagaaagatgggatatagaagcaataaacgatataccagaatacatgagggttatttacataggtcttcttggtatgttcgaggagtacaaggaaaatcttataaatgcagggggaagagaatattgtttgtattacgctattgaagttatgaaagagataattcgttgctatcagaaggaggccgaattcttccataaagggtacgtccctgggtatgaagagtacatggaaaactctgtgatatctggtggctacaaaatgtttataatcttggaagttattacgagaggcgaatttggtgtaaaagaaacgcttgactgggcgatgggtgttcctgaaatggtaaaatcgtctagtctaattgccagattcgtcgatgatgttcaaacttataaagccgaagaagagagaggagaagtggtaagcgcgattaggtgctatatgaaagattatggtgtttctgaagaggaagcgtgcggcaaaatgagagaaatgattgaaatggagtggcaaaagctaaataaggagacccaagaaaggaggaacgaggtatccgctccagtagttatccccagtttaaatttcacgagagtacttgaagttatgtatgaccaaggagacgggtactctgactctcaaggcgttactaaagacaggattacagccttgcttagacatcctatcgaaatatga

SEQ ID NO: 8 - protein, 548 aa, synthetic construct
  REGION 1..548: note = SchTPS3 - truncation
MSSTKIESASDDASRRDANFHPTVWGDFFLNLSSNFLENNKDVQEKHEKLREEVKKLVVDSADDIPSKIQLIDDIIRLGVEYHFEEEIKAELEKLHDNQLHLNFDLATTSTWLRLLRTHGFSVSSDVFKRFKNANGEFESEDARGWWCLYEATHLRVPDDKILEEALQVSRKKLEALLTELTFPLKECVRNALHIPYYRSVPRLEARQYIPSYDAQPTKIESLSLFAKIDFNMLQALHQIELREVSRWWKDFDFSNKLPYARDRIAEGYYWIMGAHHEPKFSLSRKFLTKVIGITSLIDDTYDVYGSLEEVTLFTKAVERWDIEAINDIPEYMRVIYIGLLGMFEEYKENLINAGGREYCLYYAIEVMKEIIRCYQKEAEFFHKGYVPGYEEYMENSVISGGYKMFIILEVITRGEFGVKETLDWAMGVPEMVKSSSLIARFVDDVQTYKAEEERGEVVSAIRCYMKDYGVSEEEACGKMREMIEMEWQKLNKETQERRNEVSAPVVIPSLNFTRVLEVMYDQGDGYSDSQGVTKDRITALLRHPIEI